(19) United States
(12) Patent Application Publication
(10) Pub. No.: US 2011/0081379 A1
(43) Pub. Date: Apr. 7, 2011

(54) PURIFICATION METHOD AND METHOD OF PRODUCING VACCINE

(75) Inventors: Yae KUROSAWA, Asaka-shi (JP); Shintaro KOBAYASHI, Saitama (JP)

(73) Assignee: HOYA CORPORATION, Tokyo (JP)

(21) Appl. No.: 12/894,291

(22) Filed: Sep. 30, 2010

(30) Foreign Application Priority Data
Oct. 7, 2009 (JP) 2009-233759
Jul. 5, 2010 (JP) 2010-153160

(51) Int. Cl. A61K 39/12; A61P 31/12

(52) U.S. Cl. 424/218.1

(57) ABSTRACT

A method of purifying a virus or viral antigen from a sample solution containing the virus or viral antigen is provided. The method comprises: preparing sintered powder of hydroxyapatite, wherein a specific surface area of particles of the sintered powder is in the range of 2.0 to 11.0 m²/g; bringing the sample solution into contact with the sintered powder to thereby adsorb the virus or viral antigen to the particles of the sintered powder; and supplying an eluant to the sintered powder to thereby elute the adsorbed virus or viral antigen from the particles of the sintered powder. The method is capable of purifying the virus from the sample solution with an uniform yielding ratio and good repeatability. Furthermore, a method of producing a vaccine of the virus or viral antigen by using the method of purifying the virus or viral antigen is provided.

### FIELD OF THE INVENTION

The present invention relates to a method of purifying a virus or viral antigen from a sample solution and a method of producing a vaccine by using such a purification method.

### BACKGROUND ART

A virus or viral antigen (hereinafter, referred to as “viruses”) are separated and purified from a sample solution containing a culture solution containing the viruses and host cells. Such separation and purification processes are important processes in a field of biogenetics, clinical diagnosis and production of vaccines.

Generally, the viruses do not exist alone in the sample solution described above, but the viruses exist with cultivated cells, contaminating proteins and the like in the sample solution. Therefore, it is necessary to separate and purify the viruses from the sample solution.

Heretofore, separation and purification processes for such viruses have been performed by an ultra centrifugation, a density gradient centrifugation method and the like. However, these methods require expensive and large apparatuses in addition to complicated operations. Therefore, works for the separation and purification processes were very complicated.

In order to solve such problems, it has been proposed that sintered powder of hydroxyapatite is used for an adsorbent provided in a separation apparatus as a method of easily separating and purifying viruses from a sample solution containing the viruses for a short period of time without reducing activity of the viruses (JP-A 2000-262280 is an example of a related art).

However, in such a method, an uniform yielding (collection) ratio of the viruses to be purified cannot be obtained and further the adsorbent does not have high durability. Therefore, there is a problem in that the viruses are not purified with good repeatability.

It is an object of the present invention to provide a method of purifying viruses from a sample solution containing the viruses with an uniform yielding ratio and good repeatability (hereinafter, simply referred to as “separation method”). Furthermore, it is another object of the present invention to provide a method of producing a vaccine by using such a separation method.

These objects are achieved by the present inventions (1) to (7) described below.

(1) A method of purifying a virus or viral antigen from a sample solution containing the virus or viral antigen, the method comprising: preparing sintered powder of hydroxyapatite and the sintered powder including particles, wherein a specific surface area of the particles of the sintered powder is in the range of 2.0 to 11.0 m²/g; bringing the sample solution into contact with the sintered powder to thereby adsorb the virus or viral antigen to the particles of the sintered powder; and supplying an eluant to the sintered powder to thereby elute the adsorbed virus or viral antigen from the particles of the sintered powder.

This makes it possible to separate and purify viruses from a sample solution containing the viruses with an uniform yielding ratio and good repeatability.

In the method described in the above-mentioned item (1), a porosity in surfaces of the particles of the sintered powder is in the range of 0.1 to 0.14 μm.

According to the method, it is possible to selectively adsorb the viruses which are in contact with sintered powder without adsorbing foreign substances other than the viruses. Therefore, it is possible to separate and purify viruses from the sample solution with excellent accuracy.

In the method described in the above-mentioned item (1), a voidage in the surfaces of the particles of the sintered powder is in the range of 10 to 35%.

According to the method, it is possible to selectively adsorb the viruses which are in contact with sintered powder without adsorbing foreign substances other than the viruses. Therefore, it is possible to separate and purify viruses from the sample solution with excellent accuracy.

(2) In the method described in the above-mentioned item (1), an average particle diameter of the particles of the sintered powder is in the range of 10 to 100 μm.

According to the method, it is possible to improve a ratio of filling sintered powder to an adsorbent filling space which is provided in a separation apparatus to separate the viruses. Therefore, there are many opportunities that the viruses are brought into contact with the sintered powder, so that it is possible to reliably separate and purify the virus from the sample solution.

(3) In the method described in the above-mentioned item (1), the preparing sintered powder includes: mixing raw materials to obtain a slurry containing primary particles of the hydroxyapatite and aggregates of the primary particles; drying the slurry to obtain secondary particles of the hydroxyapatite; and sintering the secondary particles of the hydroxyapatite to obtain the sintered powder.

Use of such sintered powder makes it possible to allow a specific surface area thereof to fall within the above range with ease.

(4) In the method described in the above-mentioned item (3), the secondary particles are obtained by granulating the primary particles of the hydroxyapatite and the aggregates of the primary particles.

Use of such sintered powder makes it possible to allow a specific surface area thereof to fall within the above range with ease.

(5) In the method described in the above-mentioned item (1), the eluant is a phosphate-based buffer solution.

This makes it possible to prevent the viruses to be separated from being altered.

(6) In the method described in the above-mentioned item (1), the viruses include a virus belongs to Flaviviridae.

When the virus which belongs to such a family is separated and purified, the separation method according to the present invention is preferably used.

(7) A method of producing a vaccine of the virus or viral antigen by using the method of purifying the virus or viral antigen described (1).

This makes it possible to purify the viruses without reduction of infectivity titer thereof.

According to the separation method according to the present invention, it is possible to separate and purify viruses from a sample solution containing the viruses with an uniform yielding ratio and good repeatability. Furthermore, since the viruses, which have been separated and purified by the separation method according to the present invention, maintains a good bioactivity, it is possible to apply the method to a method of producing a vaccine having excellent safety and availability.

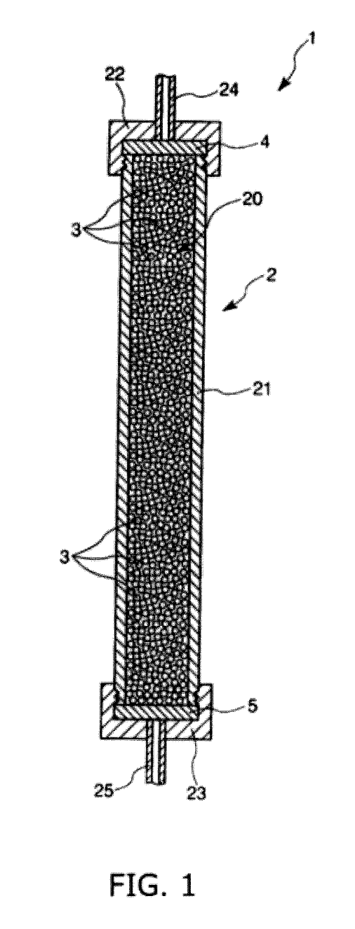

FIG. 1 is a vertical section view which shows one example of a separation apparatus to be used for a separation method according to the present invention.

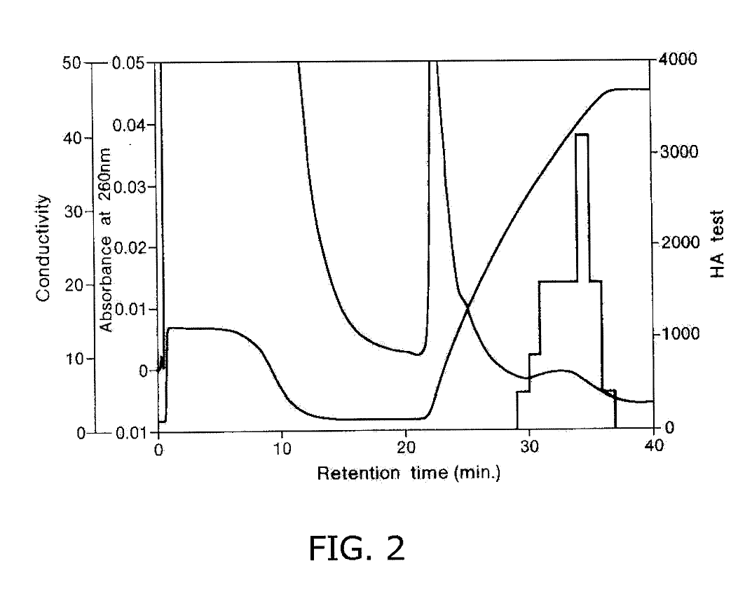

FIG. 2 shows an elution pattern of dengue virus which is obtained in Example 1.

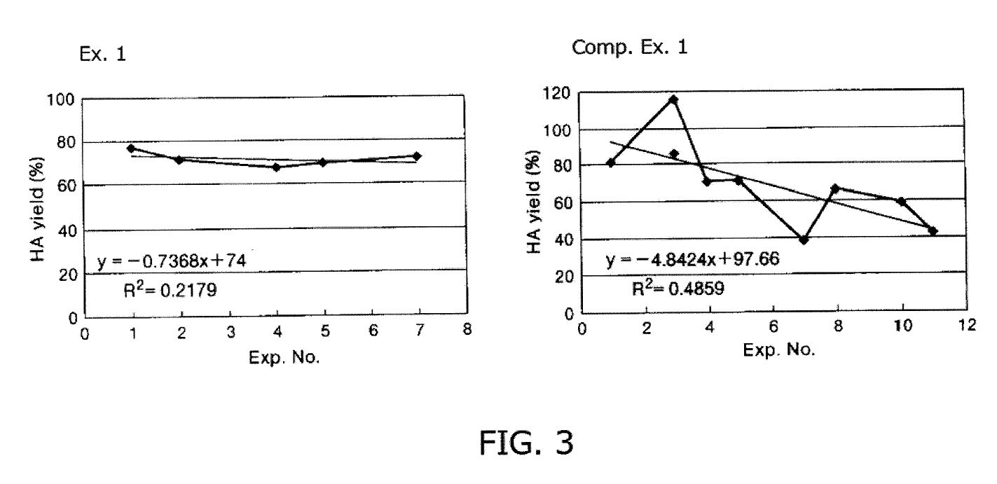

FIG. 3 shows a yielding ratio of dengue virus collected when the separation methods in Example 1 and Comparative Example 1 are performed repeatability.

Hereinbelow, a separation method and a method of producing a vaccine according to the present invention will be described in detail based on a preferred embodiment.

First, prior to the description of the separation method and the method of producing the vaccine according to the present invention, one example of a separation apparatus (adsorption apparatus) to be used for the separation method according to the present invention will be described.

FIG. 1 is a vertical section view which shows one example of a separation apparatus to be used for a separation method according to the present invention. It is to be noted that in the following description, the upper side and the lower side in FIG. 1 will be referred to as “inflow side” and “outflow side”, respectively.

More specifically, the inflow side means a side from which liquids such as a sample solution (i.e., a liquid containing a virus or viral antigen) and an elution liquid are supplied into the separation apparatus to separate (purify) a target virus or viral antigen (hereinafter, simply referred to as “viruses”), and the outflow side means a side located on the opposite side from the inflow side, that is, a side through which the liquids described above discharge out of the separation apparatus as a discharge liquid.

The separation apparatus 1 shown in FIG. 1, which separates (isolates) a target virus (isolation material) from the sample solution, includes a column 2, a granular adsorbent (filler) 3, and two filter members 4 and 5.

The column 2 is constituted from a column main body 21 and caps 22 and 23 to be attached to the inflow-side end and outflow-side end of the column main body 21, respectively.

The column main body 21 is formed from, for example, a cylindrical member. Examples of a constituent material of each of the parts (members) constituting the column 2 including the column main body 21 include various glass materials, various resin materials, various metal materials, and various ceramic materials and the like.

An opening of the column main body 21 provided on its inflow side is covered with the filter member 4, and in this state, the cap 22 is threadedly mounted on the inflow-side end of the column main body 21. Likewise, an opening of the column main body 21 provided on its outflow side is covered with the filter member 5, and in this state, the cap 23 is threadedly mounted on the outflow-side end of the column main body 21.

The column 2 having such a structure as described above has an adsorbent filling space 20 defined by the column main body 21 and the filter members 4 and 5, and at least a part of the adsorbent filling space 20 is filled with the adsorbent 3 (in this embodiment, almost the entire of the adsorbent filling space 20 is filled with the adsorbent 3).

A volumetric capacity of the adsorbent filling space 20 is appropriately set depending on the volume of a sample solution to be used and is not particularly limited, but is preferably in the range of about 0.1 to 100 mL, and more preferably in the range of about 1 to 50 mL per 1 mL of the sample solution.

By setting a size of the adsorbent filling space 20 to a value within the above range and by setting a size of the adsorbent 3 (which will be described later) to a value within a range as will be described later, it is possible to selectively isolate (purify) a target virus from the sample solution, that is, reliably separate the virus from foreign substances other than the viruses contained in the sample solution.

Further, in the column 2, liquid-tightness between the column main body 21 and the caps 22 and 23 is ensured by attaching the caps 22 and 23 to the column main body 21.

An inlet pipe 24 is liquid-tightly fixed to the cap 22 at substantially the center thereof, and an outlet pipe 25 is also liquid-tightly fixed to the cap 23 at substantially the center thereof. The sample solution (liquid) described above is supplied to the adsorbent 3 through the inlet pipe 24 and the filter member 4. The sample solution supplied to the adsorbent 3 passes through gaps between particles of the adsorbent 3 and then discharges out of the column 2 through the filter member 5 and the outlet pipe 25. At this time, the virus and the foreign substances other than the viruses contained in the sample solution (sample) are separated based on a difference in degree of adsorption between the virus and the foreign substances with respect to the adsorbent 3 and a difference in degree of affinity between the virus and the foreign substances with respect to the elution liquid.

Each of the filter members 4 and 5 has a function of preventing the adsorbent 3 from discharging out of the adsorbent filling space 20. Further, each of the filter members 4 and 5 is formed of a nonwoven fabric made of a synthetic resin such as polyurethane, polyvinyl alcohol, polypropylene, polyetherpolyamide, polyethylene terephthalate, and polybutylene terephthalate; a foam (a sponge-like porous body having communicating pores); a woven fabric; a mesh; sintered glass filter; or the like.

In the separation method according to the present invention, features reside in configurations of the adsorbent 3 described above. Hereinafter, a description will be made on this adsorbent 3 in detail.

The adsorbent 3 is sintered powder of hydroxyapatite. A specific surface area of the adsorbent 3 is in the range of 2.0 to 11.0 m²/g.

In order to selectively separate and purify the target virus from proteins and foreign substances derived from the host cells, which are contained in the sample solution containing the culture solution and the host cells, it is necessary to selectively adsorb the viruses to the adsorbent (sintered powder) 3. In this regard, by study of the present inventors, it has found that the specific surface area of the adsorbent (sintered powder) 3 is greatly involved in adsorption of the viruses. In other words, it has found that the viruses can be selectively adsorbed to the adsorbent 3 by setting the specific surface area thereof to a value within a predetermined range. This is because the specific surface area means a size of a surface where the adsorbent 3 brings into contact with the viruses, namely an opportunity when the adsorbent 3 brings into contact with the viruses.

The present inventors have further studied such points, so that it has found that the viruses are selectively adsorbed to the adsorbent 3 by setting the specific surface area of the adsorbent 3 to a value within the range of 2.0 to 11.0 m²/g. In addition, it has found that the virus is separated and purified from the sample solution containing the viruses with an uniform yielding (collection) ratio and good repeatability. Consequently, the present inventors have accomplished the present invention.

In this present invention, the sintered powder of hydroxyapatite (Ca₁₀(PO₄)₆(OH)₂) is powder obtained by sintering secondary particles of hydroxyapatite. The secondary particles of hydroxyapatite are dried powder obtained by drying a slurry containing primary particles of hydroxyapatite and aggregates thereof and granulating them. A Ca/P ratio of hydroxyapatite is intended to be in the range of about 1.64 to 1.70. Since such sintered powder and secondary particles of hydroxyapatite have chemically a stable apatite-structure, the sintered powder obtained by sintering the secondary particles can be reliably utilized to the adsorbent 3 which is provided with the separation apparatus.

When the sample solution containing the culture solution and the host cells is supplied to the adsorbent 3 having such a configuration, the virus contained in the sample solution specifically adsorbs to the adsorbent 3 having the specific surface area in the range of 2.0 to 11.0 m²/g with inherent adsorbability (supporting power). The virus is separated from the foreign substances other than the viruses contained in the sample solution and purified according to a difference between the adsorbabilities of the virus and the foreign substances.

The specific surface area of the adsorbent (sintered powder of hydroxyapatite) 3 may be in the range of 2.0 to 11.0 m²/g, but is preferably in the range of about 6.0 to 11.0 m²/g. If the specific surface area falls within the above range, the viruses selectively can adsorb to the adsorbent 3. Therefore, it is possible to separate and purify the virus from the sample solution with excellent accuracy. Generally, as the specific surface area of the adsorbent 3 becomes large, adsorbed amounts of not only the virus but also the foreign substances are improved. As a result, it is difficult to separate and purify only virus with the excellent accuracy. However, the present invention sets the specific surface area of the adsorbent 3, to which the foreign substances hardly adsorb as much as possible, while sufficiently exhibiting adsorption capability of hydroxyapatite with respect to the virus.

Furthermore, a porosity (average pore size) in a surface of the adsorbent (sintered powder) 3 is preferably in the range of about 0.1 to 0.14 μm, more preferably in the range of about 0.11 to 0.14 μm, and even more preferably in the range of about 0.12 to 0.13 μm. In this regard, it is to be noted that the porosity (pore size) of the adsorbent 3 indicates a surface shape of the adsorbent 3, namely irregularity of the surface of the adsorbent 3. By setting the porosity of the adsorbent 3 to a value within the above range, the virus falls into pores, so that elution of the virus becomes late. This makes it possible to separate the virus from contaminating proteins other than the virus with good accuracy. Accordingly, it is possible to separate and purify the virus from the sample solution with more excellent accuracy.

Furthermore, a voidage in the surface of the adsorbent (sintered powder) 3 is preferably in the range of about 10 to 35%, and more preferably in the range of about 25 to 35%. The voidage is another index which is different from the porosity indicating the surface shape of the adsorbent 3. By setting the voidage of the adsorbent 3 to a value within the above range, a frequency of times that the virus falls into the pores is improved to thereby delay the elution of the virus. This makes it possible to separate and purify the virus from the sample solution with excellent accuracy. In this regard, in the case where the voidage exceeds the upper limit value noted above, there is a fear that mechanical strength of the adsorbent becomes insufficient.

Furthermore, an average particle diameter of the adsorbent 3 is preferably in the range of about 10 to 100 μm, and more preferably in the range of about 40 to 80 μm. By using the sintered powder having such an average particle diameter as the adsorbent 3, it is possible to improve a ratio of filling the adsorbent 3 to the adsorbent filling space 20. Therefore, since opportunities that the virus brings into contact with the adsorbent 3 increase, so that it is possible to reliably separate and purify virus from the sample solution.

In this regard, the separation apparatus 1 is described about a case that almost the entire of the adsorbent filling space 20 is filled with the adsorbent 3 as in the case of this embodiment. Alternatively, the separation apparatus 1 may be described about a case that the adsorbent filling space 20 may be partially filled with the adsorbent 3 (e.g., a part of the adsorbent filling space 20 located on its one side where the inlet pipe 24 is provided may be filled with the adsorbent 3). In this case, the remaining part of the adsorbent filling space 20 may be filled with another adsorbent.

The adsorbent 3 (sintered powder) as described above can be produced by, e.g., the following method.

A method of producing the sintered powder according to the present embodiment includes three steps.

A first step [S1] is a step of reacting a calcium compound and a phosphate compound while stirring a mixture of a first liquid containing the calcium compound (calcium source) such as calcium hydroxide and a second liquid containing the phosphate compound (phosphate source) such as phosphoric acid to obtain a slurry containing primary particles of hydroxyapatite and aggregates thereof. A second step [S2] is a step of drying the slurry containing the primary particles and the aggregates thereof and granulating them to obtain dried powder which is constituted of secondary particles of hydroxyapatite as a main component. A third step is a step of sintering the dried powder to obtain sintered powder which is constituted of hydroxyapatite.

Hereinafter, a description will be made on these steps one after another.

[S1: Step of Obtaining Slurry Containing Aggregates of Hydroxyapatite (First Step)]

First, a first liquid containing a calcium-based compound containing calcium as a calcium source is prepared.

The calcium-based compound (calcium source) is not particularly limited to a specific compound. Examples of the calcium-based compound include calcium hydroxide, calcium oxide, calcium nitrate and the like. These compounds may be used singly or in combination of two or more of them. Among them, calcium hydroxide is particularly preferred as the calcium source. This makes it possible to reliably obtain hydroxyapatite having impurities of a low amount, which is synthesized in this step. In this regard, hereinafter, it is to be noted that a description will be made on a case that calcium hydroxide is used as the calcium source as an example.

A solution and suspension containing calcium hydroxide as the calcium source can be used as the first liquid. Particularly, a calcium hydroxide suspension, in which the calcium hydroxide is suspended in water, is used preferably. If hydroxyapatite is synthesized by using such a suspension, fine primary particles of hydroxyapatite can be obtained.

Next, a second liquid (phosphoric acid-containing solution) containing phosphoric acid as a phosphate source is prepared.

A solvent for dissolving phosphoric acid is not particularly limited, and any solvent can be used as long as it does not inhibit the reaction between calcium hydroxide and phosphoric acid in this step S1. Examples of such a solvent include water, an alcohol such as methanol and ethanol, and the like. These solvents may be used in combination of two or more of them. However, among them, water is particularly preferred. If water is used as the solvent, it is possible to reliably prevent the reaction between calcium hydroxide and phosphoric acid from being interfered.

Next, the prepared first and second liquid are mixed each other to obtain a mixture. Then, calcium hydroxide and phosphoric acid are reacted with stirring the mixture to obtain a slurry containing the primary particles of hydroxyapatite and the aggregates thereof.

To be concrete, the second liquid is dropped into a dispersion liquid (first liquid) containing calcium hydroxide obtained by stirring the first liquid in a vessel (not shown). By doing so, the first liquid (dispersion liquid) and the second liquid are mixed with each other to obtain a mixture. Thereafter, calcium hydroxide is reacted with phosphoric acid in the mixture to obtain the slurry containing the primary particles of hydroxyapatite and the aggregates thereof.

In this method, used is a wet synthesis method that phosphoric acid is used as a aqueous solution as described above. This makes it possible to efficiently and easily synthesize hydroxyapatite (synthetic material) without using an expensive production facility. In the reaction between calcium hydroxide and phosphoric acid, only water is products except for hydroxyapatite. Therefore, by-products do not remain in the produced secondary particles (dried powder) and the produced sintered powder. Furthermore, since this reaction is an acid-base reaction, there is an advantage that this reaction can easily be controlled by adjusting pH of a calcium hydroxide dispersion liquid and a phosphoric acid aqueous solution.

Further, by performing this reaction with stirring, it is possible to efficiently perform the reaction between calcium hydroxide and phosphoric acid. In other words, it is possible to improve efficiency of the reaction therebetween.

Furthermore, power for stirring (stirring power) the mixture containing the first and second liquids is not particularly limited to a specific power, but preferably in the range of about 0.75 to 2.0 W and more preferably in the range of about 0.925 to 1.85 W per 1 liter of the mixture (slurry). By setting the stirring power to a value within the above range, it is possible to further improve the efficiency of the reaction between calcium hydroxide and phosphoric acid.

A content of calcium hydroxide in the first liquid is preferably in the range of about 5 to 15 wt % and more preferably in the range of about 10 to 12 Wt %. A content of phosphoric acid in the second liquid is preferably in the range of about 10 to 25 wt % and more preferably in the range of about 15 to 20 Wt %. By setting the content of each of calcium hydroxide and phosphoric acid to a value within the above range, it is possible to efficiently react calcium hydroxide and phosphoric acid. Consequently, it is possible to reliably synthesize hydroxyapatite. This is because an opportunity of contacting between calcium hydroxide and phosphoric acid increases when the second liquid is dropped into the first liquid while stirring it.

A rate of dropping the second liquid into the first liquid is preferably in the range of about 1 to 40 L/hr and more preferably in the range of about 3 to 30 L/hr. By mixing (adding) the second liquid with (to) the first liquid at such a dropping rate, it is possible to react calcium hydroxide with phosphoric acid under milder conditions.

In this case, the second liquid is preferably dropped (added) into (to) the first liquid for a length of time from about 5 to 32 hours, and more preferably for a length of time from about 6 to 30 hours. By dropping the second liquid into the first liquid in such a period of time to react calcium hydroxide with phosphoric acid, it is possible to sufficiently synthesize hydroxyapatite. It is to be noted that even if the time for dropping the second liquid into the first liquid is prolonged to exceed the above upper limit value, it cannot be expected that the reaction between calcium hydroxide and phosphoric acid will further proceed.

When the reaction between calcium hydroxide and phosphoric acid gradually proceeds, the primary particles of hydroxyapatite (synthetic material) are produced in the slurry (hereinafter, simply referred to as “primary particles”). A chemical structure of such primary particles includes positively-charged parts and negatively-charged parts. Therefore, Van der Waals' forces (intermolecular force) are made between the positively-charged parts in the chemical structure of one primary particle of the primary particles and the negatively-charged parts in the chemical structure of the other primary particle of the primary particles. By this Van der Waals' forces, the one primary particle and the other primary particle adhere to each other to obtain a pre-aggregate. Then, in the surly, pre-aggregates are agglutinated to obtain aggregates of hydroxyapatite (synthetic material) (hereinafter, simply referred to as “aggregates”). The aggregates make a viscosity of the slurry increase gradually.

[S2: Step of Obtaining Secondary Particles of Hydroxyapatite by Drying Slurry (Second Step)

In this second step, by drying the slurry containing the primary particles of hydroxyapatite and the aggregates thereof obtained in the step [S1] and granulating them, dried powder constituted of the secondary particles of hydroxyapatite is obtained as a main component thereof.

A method of drying the slurry is not particularly limited to a specific method, but a spray drying method is preferably used. Accordingly to such a method, the primary particles of hydroxyapatite and the aggregates thereof are granulated, so that it is possible to reliably obtain powder having a predetermined particle diameter for a short period of time.

Furthermore, a drying temperature of the slurry is preferably in the range of about 75 to 250° C. and more preferably in the range of about 95 to 220° C. By setting the drying temperature to a value within the above range, it is possible to reliably obtain the secondary particles (dried powder) having an uniform particle diameter.

[S3: Step of Sintering Secondary Particles to Obtain Sintered Powder of Hydroxyapatite (Third Step)]

In the third step, by sintering the dried powder of hydroxyapatite obtained in the step [S2], sintered powder constituted of hydroxyapatite is obtained as a main component thereof. Compressive particle strength (break strength) of such sintered powder is improved as compared with that of the dried powder.

In this case, a sintering temperature of the powder is preferably in the range of about 800 to 1100° C. and more preferably in the range of about 900 to 1000° C.

In this regard, the method of producing the sintered powder according to the present embodiment, in particular, is suitable for the production of sintered powder having an intended particle diameter in the range of about 10 to 100 μm.

By completing the steps as described above, it is possible to produce the sintered powder of hydroxyapatite. In this regard, the specific surface area of the adsorbent (sintered powder) 3 used for the purification method according to the present invention is in the range of 2.0 to 11.0 m²/g as described above. In the method of producing the sintered powder described above, the specific surface area of the adsorbent 3 can be easily set to a value within the above range by appropriately adjusting the sintering temperature in the step [S3], dispersabilities of the primary particles and the aggregates thereof in the step [S1] (particle distribution thereof) and the like.

Furthermore, the porosity of the adsorbent (sintered powder) 3 is preferably in the range of 0.1 to 0.14 μm, and the voidage thereof is preferably in the range of 10 to 35%. In the method of producing the sintered powder described above, each of such a porosity and voidage of the adsorbent 3 can be easily set to a value within the above range by appropriately adjusting the sintering temperature in the step [S3], the dispersabilities of the primary particles and the aggregates thereof in the step [S1] (particle distribution thereof), the drying temperature in the step [S2] and the like.

In this regard, the adjustment of the dispersabilities (particle distribution) of the primary particles and the aggregates thereof in the step [S1] can be performed by appropriately adjusting, e.g., a power of stirring the mixture of the first and second liquids and a temperature of the mixture. Alternatively, the adjustment can also be performed by physically pulverizing the produced aggregates of the primary particles and then dispersing the pulverized aggregates into the slurry.

Furthermore, a method of physically pulverizing the aggregates of the primary particles of hydroxyapatite is not particularly limited to a specific method. Examples of the method include: a wet jet-mill method of crashing droplets of the sprayed slurry under a high pressure; a ball mill method of placing the slurry and balls constituted of ceramics such as zirconia into a closed container and rotating the closed container; and the like.

Next, a method of purifying a virus or viral antigen by using the separation apparatus 1 as described above (i.e., the purification method according to the present invention) will be described.

(1) Preparation Step

First, a sample solution containing a culture solution and host cells is prepared.

Here, viruses are obtained by allowing to grow in a culture cell in addition to a brain cell and a nerve cell of a mammal derived from animals and an ovum gallinaceum. Therefore, one containing the culture solution allowed them to grow and the host cells is used as the sample solution containing the viruses.

The viruses are not particularly limited to a specific virus, but include viruses having envelopes, no envelope and the like. Examples of a family of the viruses having the envelopes include: Flaviviridae to which dengue virus and Japanese encephalitis virus belong; Orthomyxoviridae to which flu virus belongs; Togaviridae to which rubella virus belongs; Paramyxoviridae to which measles virus and mumps virus belong. Examples of a family of the viruses having no envelopes include: Papillomaviridae to which papilloma virus belongs; Reoviridae to which reovirus and rotavirus belong. Among these viruses, one which belongs to Flaviviridae is preferable. A diameter (size) of the dengue virus and the Japanese encephalitis virus, which belong to Flaviviridae, is in the range of about 40 to 50 nm. When the virus having such a size is separated and purified, it is possible to reliably separate the virus and other foreign substances contained in the sample solution with higher accuracy by using the adsorbent 3 as described above.

Examples of the viral antigen includes: ones having no toxicity or low toxicity as a virus; ones in which parts exhibiting antigenecity have been selectively cut from viruses; and the like.

(2) Supplying Step (First Step)

Next, the prepared sample solution is supplied to the adsorbent 3 through the inlet pipe 24 and the filter member 4 to be in contact with the adsorbent 3 and to pass through the column 2 (separation apparatus 1).

Therefore, the viruses having high adsorption capability with respect to the adsorbent 3 and foreign substances having the relatively high adsorption capability with respect to the adsorbent 3 among the foreign substances other than the viruses are carried on the adsorbent 3 in the column 2. The foreign substances having low adsorption capability with respect to the adsorbent 3 is discharged out of the column 2 through the filter member 5 and the outlet pipe 25.

(3) Fractionation Step (Second Step)

Next, a phosphate elution buffer as an eluate is supplied into the column 2 through the inlet pipe 24 and the filter member 4 to elute the adsorbed viruses.

Thereafter, the eluant discharged out of the column 2 through the outlet pipe 25 and the filler member 5 is fractionated (collected) to obtain fractions having a predetermined amount of the eluant. In this way, the viruses, which are adsorbed to the adsorbent 3, and other foreign substances are collected (separated from each other) to the fractions in state that they are eluted, depending on the difference between absorbability of the viruses with respect to the adsorbent 3 and absorbability of their foreign substances with respect to the adsorbent 3.

Examples of the phosphate elution buffer include sodium phosphate, potassium phosphate, lithium phosphate and the like.

A pH of the phosphate elution buffer is not particularly limited, but is preferably in a neutral region, concretely, preferably in the range of about 6 to 8, and more preferably in the range of about 6.5 to 7.5. This makes it possible to prevent the virus to be separated from being altered, thereby preventing biological activity of the virus from standing a loss. Furthermore, it is also possible to reliably prevent the adsorbent 3 from being altered, so that it is also to prevent separation capacity of the separation apparatus 1 from being changed.

Therefore, by using the phosphate elution buffer of which pH falls within the above noted ranges, it is possible to improve a yielding ratio of a target virus.

Furthermore, a salt concentration of the phosphate elution buffer is preferably about 600 mM. The separation of the virus by using the phosphate elution buffer having such a salt concentration makes it possible to prevent adverse affects from occurring to the virus due to existence of metal ions in the phosphate elution buffer.

Specifically, the salt concentration of the phosphate elution buffer is preferably in the range of about 1 to 600 mM. Further, it is preferred that the salt concentration of the phosphate elution buffer is changed in a continuous manner or a stepwise manner when a separate operation of the virus. This makes it possible to efficiently improve the separate operation of the virus.

A flow rate of the phosphate elution buffer to flow in the adsorbent filling space 20 is preferably in the range of about 0.1 to 10 mL/min, and more preferably in the range of about 1 to 5 mL/min. By separating the virus at such a flow rate, it is possible to reliably separate a target virus from the sample solution without a long time to be needed to the separation operation. That is to say, it is possible to obtain the virus having high purity. By the operations as described above, the virus is collected to a predetermined of fractions.

Furthermore, the target virus is purified by using the purification method according to the present invention (purification step), and thereafter a vaccine can be produced by inactivating the purified virus (inactivating step). According to such a method of producing the vaccine, since the virus is purified with high purity, it is possible to greatly reduce a risk of contamination due to other microbes. As a result, it is possible to produce a vaccine having high safety.

In this inactivating step, various kinds of methods can be selected as a method of inactivating the virus, though depending on a kind of vaccine to be produced.

Although the purification method and the method of producing the vaccine according to the present invention have been described above, the present invention is not limited thereto.

For example, the purification method according to the present invention may further include a pre-step before the step [S1], an intermediate step between the step [S1] and the step [S2] or between the step [S2] and the step [S3], and a post-step after the step [S3] for arbitrary purposes.

### EXAMPLES

Next, the present invention will be described with reference to specific examples.

### 1. Purification of Dengue Virus

### Example 1

—1— First, a sample solution was prepared as follows: The dengue virus was allowed to grow with a C6/36 cell derived from a mosquito to obtain a culture supernatant, and thereafter the culture supernatant was extracted. The extracted culture supernatant was filtered by a filter having a filter size of 0.22 μm to obtain the sample solution.

—2— Next, 10 mL of the sample solution (sample) was supplied (applied) into a separation apparatus to adsorb the virus and foreign substances to an adsorbent. Then, an eluate A and an eluate B were supplied into the separation apparatus at a flow rate of 1 mL/min for 15 minutes so that an amount ratio of the eluate B was continuously changed in the range of 0 to 100%. Thereafter, the eluate B was supplied into the separation apparatus at the flow rate of 1 mL/min. Then, an eluant containing the virus and the foreign substances discharged out of the column. The discharged eluant was fractionated in fractions of a fraction number 1 to 10 by 2 mL and in fractions of the fraction number 11 to 30 by 1 mL.

As a result, the dengue virus contained in the sample solution could be separated from the foreign substances as shown in FIG. 2. Specifically, the dengue virus could be separated from the foreign substances, which discharged in the fractions before 20 minutes of the retention time in FIG. 2, and the foreign substances having low adsorbability, which discharged in the fractions from 20 minutes of the retention time in FIG. 2. That is to say, the dengue virus could be collected (purified) in the fractions containing the eluant which discharged after around the 30 minutes of the retention time.

It is to be noted that 10 mM phosphate elution buffer (pH 7.2) was used as the eluate A and 600 mM phosphate elution buffer (pH 7.2) was used as the eluate B.

In this regard, it is to be noted that a column (size 4.6 mm×35 mm) in which about 0.6 g of hydroxyapatite beads (sintered powder of which average particle diameter was 40 μm) produced as described below as the adsorbent was filled into the adsorbent filling space was used in the separation apparatus.

—2A— First, calcium hydroxide was suspended in pure water to obtain a calcium hydroxide suspension, and then an aqueous phosphoric acid solution was dropped into the calcium hydroxide suspension to obtain a mixture. The mixture was stirred with a stirring power of 1 kW at a temperature of 30° C. for 24 hours. In this way, 500 L of a slurry containing 10 wt % of primary particles of hydroxyapatite was obtained.

It is to be noted that the thus obtained synthesis material was found to be hydroxyapatite by powder X-ray diffractometry.

—2B— Next, the slurry containing the primary particles of hydroxyapatite was spray-dried at 150° C. using a spray drier (manufactured by OHKAWARA KAKOHKI Co., Ltd. under the trade name of “OC-20”) to thereby obtain particulate dried powder.

—2C— Furthermore, parts of the dried powder were classified to obtain particles having a median particle diameter of about 40 μm. Thereafter, the particles were sintered in an electric furnace at a temperature of 950° C. for 4 hours to obtain sintered powder.

In this regard, an average particle diameter, a specific surface area, a porosity and a voidage of particles of the thus obtained sintered powder of hydroxyapatite were about 40 μm, 6.6 m²/g, 0.13 μm, and 32%, respectively.

### Examples 2 to 5 and Comparative Examples 1 and 2

A dengue virus contained in a sample solution was collected (separated and/or purified) in the same manner as in the Example 1 except that sintered powder produced under the conditions as shown in Table 1 was used as the sintered powder of hydroxyapatite which was used as an adsorbent.

In this regard, the purifications of the dengue virus in the Example 1 and the Comparative Example 1 were repeatedly performed five times and eight times by using the same separation apparatus as each other, respectively.

TABLE 1

| | Production conditions | | | | | Sintered powder | | | |
|---|---|---|---|---|---|---|---|---|---|
| | -2A- | | | -2B- | -2C- | Average | Specific | | |
| | | | Stirring | Drying | Sintering | particle | surface | | |
| | Temperature | Time | power | temperature | temperature | diameter | area | Porosity | Voidage |
| | [° C.] | [hr] | [kw] | [° C.] | [° C.] | [μm] | [m²/g] | [μm] | [%] |
| Ex. 1 | 30 | 24 | 1 | 150 | 950 | 40 | 6.6 | 0.13 | 32 |
| Ex. 2 | 30 | 24 | 1 | 150 | 1000 | 40 | 3.3 | 0.14 | 19 |
| Ex. 3 | 30 | 24 | 1 | 150 | 900 | 40 | 10.5 | 0.12 | 35 |
| Ex. 4 | 30 | 24 | 3 | 150 | 950 | 40 | 7.5 | 0.09 | 30 |
| Ex. 5 | 30 | 24 | 0.3 | 150 | 950 | 40 | 6.1 | 0.16 | 35 |
| Comp. ex. 1 | 30 | 24 | 1 | 150 | 700 | 40 | 21.0 | 0.1 | 50 |
| Comp. ex. 2 | 30 | 24 | 1 | 150 | 1050 | 40 | 1.2 | 0.14 | 8 |

### 2. Evaluation

### 2-1. Yielding Ratio (Purification Ratio) of Dengue Virus

In each of the Examples 1 to 5 and the Comparative Examples 1 and 2, the dengue virus contained in the fractions, in which the eluant discharged from the column after around the 30 minutes of the retention time, was subjected to a hemagglutination test (HA test) to obtain a yielding ratio of the dengue virus.

The thus obtained yielding ratio of the dengue virus in each of the Examples 1 to 5 and the Comparative Examples 1 and 2 is shown in Table 2.

TABLE 2

| | Sintered powder | | | | |
|---|---|---|---|---|---|
| | Average | Specific | | | |
| | particle | surface | | | |
| | diameter | area | Porosity | Voidage | Yielding ratio |
| | [μm] | [m²/g] | [μm] | [%] | [%] |
| Ex. 1 | 40 | 6.6 | 0.13 | 32 | 94 |
| Ex. 2 | 40 | 3.3 | 0.14 | 19 | 78 |
| Ex. 3 | 40 | 10.5 | 0.12 | 35 | 90 |
| Ex. 4 | 40 | 7.5 | 0.09 | 30 | 60 |
| Ex. 5 | 40 | 6.1 | 0.16 | 35 | 60 |
| Comp. ex. 1 | 40 | 21.0 | 0.1 | 50 | 50 |
| Comp. ex. 2 | 40 | 1.2 | 0.14 | 8 | 59 |

In the case where the dengue virus was purified by the purification method in each of the Examples, that is, the dengue virus was purified by using the sintered powder of hydroxyapatite having the specific surface area in the range of 2.0 to 11.0 m²/g as the adsorbent provided with the separation apparatus, the yielding ratio of the dengue virus was 60% or more as shown in Table 2. This shown that the dengue virus could be purified with an excellent yielding ratio. Furthermore, in each of the Examples 1 to 3, the porosity of the sintered powder was 0.12 to 0.14 μm, which shown a more excellent yielding ratio of the dengue virus.

In contrast, the yielding ratio of the dengue virus was lower than 60% in each of the Comparative Examples 1 and 2. This was because the specific surface area of the sintered powder of hydroxyapatite fell beyond the range of 2.0 to 11.0 m²/g.

### 2-2. Repeatability of Yielding Ratio of Dengue Virus

The purifications of the dengue virus in Example 1 and the Comparative Example 1 were repeatedly performed five times and eight times, respectively. In the thus obtained dengue virus contained in the fractions in which the eluant discharged from the column after around the 30 minutes of the retention time, the yielding ratio of the dengue virus was obtained by using the same method as that described in the item 2-1.

The yielding ratios of the thus obtained dengue virus in the Example 1 and the Comparative Example 1 are shown every times in FIG. 3.

As shown in FIG. 3, in the purification method of the Example 1, the dengue virus was repeatedly purified by using the same separation apparatus as each other. As a result, there was no great difference among the yielding ratios of the dengue virus in all of the times. This shown that the dengue virus could be collected with an uniform yielding ratio and good repeatability.

In contrast, in the purification method of the Comparative Example 1, the dengue virus was repeatedly purified by using the same separation apparatus as each other. As a result, the yielding ratio of the dengue virus tended to fall every times. This shown that it was impossible to collect the dengue virus with good repeatedly.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

Further, it is also to be understood that the present disclosure relates to subject matter contained in Japanese Patent Applications No. 2009-233759 (filed on Oct. 7, 2009) and No. 2010-153160 (filed on Jul. 5, 2010) and which are expressly incorporated herein by reference in its entireties.

What is claimed is:

1. A method of purifying a virus or viral antigen from a sample solution containing the virus or viral antigen, the method comprising:
preparing sintered powder of hydroxyapatite and the sintered powder including particles, wherein a specific surface area of the particles of the sintered powder is in the range of 2.0 to 11.0 m²/g;
bringing the sample solution into contact with the sintered powder to thereby adsorb the virus or viral antigen to the particles of the sintered powder; and
supplying an eluant to the sintered powder to thereby elute the adsorbed virus or viral antigen from the particles of the sintered powder.

2. The method as claimed in claim 1, wherein an average particle diameter of the particles of the sintered powder is in the range of 10 to 100 μm.

3. The method as claimed in claim 1, wherein the preparing sintered powder includes:
mixing raw materials to obtain a slurry containing primary particles of the hydroxyapatite and aggregates of the primary particles;
drying the slurry to obtain secondary particles of the hydroxyapatite; and
sintering the secondary particles of the hydroxyapatite to obtain the sintered powder.

4. The method as claimed in claim 3, wherein the secondary particles are obtained by granulating the primary particles of the hydroxyapatite and the aggregates of the primary particles.

5. The method as claimed in claim 1, wherein the eluant is a phosphate-based buffer solution.

6. The method as claimed in claim 1, wherein the viruses include a virus belongs to Flaviviridae.

7. A method of producing a vaccine of the virus or viral antigen by using the method of purifying the virus or viral antigen defined in claim 1.
